SEQUENCE LISTING (14 sequences)

SEQ ID NO: 1 - DNA, 25 bp, Artificial Sequence
  PCR primer
agtagtaggtttctgttctattggg

SEQ ID NO: 2 - DNA, 26 bp, Artificial Sequence
  PCR primer
tacttattccactgctgttattatcc

SEQ ID NO: 3 - DNA, 21 bp, Artificial Sequence
  PCR primer
gctgagtgtgttagaggtgtt

SEQ ID NO: 4 - DNA, 19 bp, Artificial Sequence
  PCR primer
aggggtgggtaggaaatgt

SEQ ID NO: 5 - DNA, 20 bp, Artificial Sequence
  PCR primer
tgtgatcttcggcaaagcct

SEQ ID NO: 6 - DNA, 20 bp, Artificial Sequence
  PCR primer
tttcctgcacccaatcttgg

SEQ ID NO: 7 - DNA, 21 bp, Artificial Sequence
  PCR primer
gctgagtgtgttagaggtgtt

SEQ ID NO: 8 - DNA, 20 bp, Artificial Sequence
  PCR primer
caggagtgggtaggaaatgc

SEQ ID NO: 9 - DNA, 20 bp, Artificial Sequence
  PCR primer
cctgccagacagtgagtctt

SEQ ID NO: 10 - DNA, 20 bp, Artificial Sequence
  PCR primer
tgggatccacctcctttaca

SEQ ID NO: 11 - DNA, 20 bp, Artificial Sequence
  PCR primer
ctgagtgtgttggaggcatc

SEQ ID NO: 12 - DNA, 21 bp, Artificial Sequence
  PCR primer
ggtgggtaggaaatgtgaggt

SEQ ID NO: 13 - DNA, 23 bp, Artificial Sequence
  PCR primer
agcttgtcatcaatggaaatccc

SEQ ID NO: 14 - DNA, 20 bp, Artificial Sequence
  PCR primer
gtgaagacgccagtggactc

(19) United States
(12) Patent Application Publication
(10) Pub. No.: US 2025/0333699 A1
(43) Pub. Date: Oct. 30, 2025

(54) METHOD FOR PREPARATION OF CYTOTOXIC T LYMPHOCYTES WITH BROAD TUMOUR-SPECIFIC REACTIVITY AND CHARACTERISTICS OF EARLY DIFFERENTIATION CELLS

(71) Applicant: CYTOVAC A/S, Hørsholm (DK)

(72) Inventors: Alexei KIRKIN, Hørsholm (DK); Karine DJANDJOUGAZIAN, Hørsholm (DK)

(21) Appl. No.: 18/573,186

(22) PCT Filed: Jun. 24, 2022
(86) PCT No.: PCT/EP2022/067303

§ 371 Date: Dec. 21, 2023

(30) Foreign Application Priority Data
Jun. 25, 2021 (EP) 21181704.4

(51) Int. Cl. C12N 5/0783; A61K 35/17; A61K 40/11; A61K 40/42; A61K 45/06

(52) U.S. Cl. CPC C12N 5/0646; A61K 35/17; A61K 40/11; A61K 40/4268; A61K 40/4269; A61K 45/06; C12N 5/0638; C12N 2501/06; C12N 2501/51; C12N 2501/515

(57) ABSTRACT

Provided is a method for preparation of a composition comprising activated human CD8⁺ lymphocytes with phenotype of stem cell-like memory cells and natural killer (NK) lymphocytes. The method entails use of short-term activation of lymphocytes by CD3/CD28 activating agents followed by treatment with DNA-demethylating agent. The invention also provides a version of the method where addition of a CD3/CD28 activating agent is made a few days after initiation of CD4⁺ mediated activation of the CD8⁺ cells; this step is also disclosed as an improvement of related methods where autologous dendritic cells have been used to activate the lymphocytes. Also provided is a method for treatment of cancer using the cells obtained from the process.

### STATEMENT REGARDING THE SEQUENCE LISTING

The instant application contains a Sequence Listing which has been submitted electronically in ASCII format and is hereby incorporated by reference in its entirety. Said ASCII copy, created on Jul. 22, 2024 is named 22922PCT00_seql_corrected.txt and is 2,903 bytes in size.

### FIELD OF THE INVENTION

The present invention relates to methods for producing effector cells that are useful in adoptive immunotherapy and also relates to the field of adoptive immunotherapy.

### BACKGROUND OF THE INVENTION

Among different approaches to cancer immunotherapy, adoptive immunotherapy shows high efficiency in inducing tumour regression in selected malignancies. Two major types of the adoptive immunotherapy are currently the most successful: CAR-T therapy and TIL therapy.

In the CAR-T approach, T lymphocytes are transfected with Chimeric Antigen Receptor (CAR), a fusion protein between an Fab antibody fragment and a fragment of a T cell receptor. This approach demonstrates high efficiency in treatment of B cell malignancies, while it as to date has proven much less effective for treatment of solid tumours.

Another approach, based on the employment of lymphocytes expanded from autologous tumours (tumour infiltrating lymphocytes, TIL), demonstrates high clinical efficiency in melanoma patients. In clinical trials employing TILs, it has been shown that the clinical effect correlates with the number of injected cells, rate of their proliferation and their lytic activity against autologous tumour cells. Unfortunately, isolation and expansion of tumour infiltrating lymphocytes with cytolytic activity against autologous tumours from other types of malignancies are significantly more difficult, restricting broad application of this technology.

It has been demonstrated in several studies that the clinical efficacy of adoptively transferred lymphocytes correlates with the presence in the injected material of T lymphocytes in their early differentiation states, such as stem cell-like memory T lymphocytes (T_{SCM}) and central memory T lymphocytes (T_{CM}) (reviewed by Gattinoni et al. 2017). Such T lymphocytes have high levels of expression of surface molecules CD62L (also known as L-selectin), CCR7 (receptor for chemokines CCL19 and CCL21), and CD27. The first two molecules are responsible for trans-endothelial traffic of lymphocytes through high endothelial venules (HEV), the structures characteristic for lymph nodes and also found in several tumours. Expression of CD27 correlates with long persistence of the injected cells in the organism. It is of note that the current methods of preparation of lymphocytes are not able to produce T lymphocytes with high levels of expression of the indicated molecules, pointing to an urgent need for improvement of the cultivation strategy in order to increase the quality of the transferred lymphocytes.

The present inventors have recently developed a method of adoptive immunotherapy of cancer, based on generation of cytotoxic lymphocytes specifically targeting a broad spectrum of cancer-testis antigens, which constitutes a group of shared tumour antigens appearing in tumour cells as a result of genome wide DNA de-methylation (Kirkin et al., 2018; WO 2008/081035). This existing procedure consists of four steps (see also FIG. 11A):
1) Generation of monocyte-derived mature dendritic cells;
2) Induction of proliferation of predominantly CD4+ cells after co-culture of the mature dendritic cells and peripheral blood lymphocytes (PBLs);
3) Treatment of the activated CD4-enriched lymphocytes with a DNA de-methylating agent leading to induction of expression of a broad spectrum of cancer-testis antigens (CTA); and
4) Generation of cytotoxic lymphocytes (via an “immunization/expansion step”) after co-culture of DNA de-methylated activated CD4+ enriched lymphocytes with unstimulated PBLs.

This method is universal and can be applied to treatment of many types of malignancies. In the present application, this process is generally termed the ALECSAT (Autologous Lymphoid Effector Cells Specific Against Tumor cells) process, more specifically the “ALECSAT-1 process”.

The present inventors have already demonstrated efficacy of this approach in treatment of patients with relapsed glioblastoma (Kirkin et al., 2018). Here, 3 out of 10 patients who received the planned 3 injections of the therapeutic cells exhibited tumour regression.

A later developed improvement of the method originally disclosed in WO 2008/081035 is disclosed in WO 2020/208054; here a portion of the mature dendritic cells used as feeder cells in the co-culture step at a very early stage, leading to a pronounced acceleration of the process in general and to an increase in the final number of effector cells. This improved process is termed “ALECSAT-2” herein.

The lack of the response in some patients may be related to insufficient induction of CTA in CD4⁺-enriched lymphocytes, as well as to insufficient expression of CCR7, CD62L and CD27 molecules by the injected cells, the molecules responsible for lymphocyte recirculation and long-term persistence in the organism. In addition, the whole process is time-consuming and rather complicated and further depends on properties of at least two cell populations in the starting material: monocytes (the source of dendritic cells) and lymphocytes. Therefore, further improvement of this technology leading to simplification of the whole process and to generation of significant number of cells of early differentiation state is desirable.

### OBJECT OF THE INVENTION

It is an object of embodiments of the invention to provide a method for generation of cytotoxic t lymphocytes with broad tumour-specific reactivity and characteristics of early differentiation cells and at a reduced time of production compared to existing protocols. It is a further object of embodiments of the present invention to provide improved methods of adoptive T-cell therapy.

### SUMMARY OF THE INVENTION

In the protocol for generation of effector cells described by Kirkin et al., 2018, generation of a CD4⁺-enriched population of lymphocytes to later be employed as antigen presenting cells for induction of CTLs has a total duration of 13 days. The resulting proliferating culture of lymphocytes is treated with a DNA-demethylating reagent to induce expression of CTA.

In the original experiments aiming to decrease total time of the protocol through decreasing time of co-culture of dendritic cells and lymphocytes, it was observed (unpublished data) that levels of expression of individual CTA were significantly increased with decreasing time of co-culture. However, this interesting observation cannot be implemented in the protocol, since reduction of the co-culture period was shown to also significantly decrease the proportion and total number of activated lymphocytes, thus rendering it impossible to generate a sufficient number of activated lymphocytes for use in the next steps of the protocol.

In the search for alternative possibilities of activation of lymphocytes, it was attempted by the inventor to target CD3 and CD28 with an activation reagent (CD3/CD28 Dynabeads, superparamagnetic beads coupled to anti-CD3 and anti-CD28 antibodies); CD3/CD28 have been used as targets in the field for decades using antibodies specific for these two cluster of differentiation proteins (Martin et al. 1986). Despite the well-known ability of targeting of this combination to induce polyclonal activation and proliferation of T lymphocytes (Levine B L et al. 1996; Kalamasz et al., 2004), it has not been reported that lymphocytes activated in this way can be induced to express cancer-testis antigens by treatment with DNA demethylating agents. However, the present inventor surprisingly found that that treatment of activated lymphocytes over a short period (≈3 days) with beads coupled to anti-CD3 and anti-CD28 antibodies induced expression of MAGE antigens, the most known members of cancer-testis antigens. Consequently, the duration of the process for preparing cytotoxic T lymphocytes “immunized” with CTA can be reduced by 10 days.

Another finding concerns the establishment of conditions promoting generation of lymphocytes with phenotypes characteristic of early differentiation-stem cell memory T lymphocytes. The starting point for this is the fact that stem cell memory T lymphocytes described first by Luca Gattinoni (2011) have many properties in common with the “classical” stem cells like embryonic stem cells or induced pluripotent stem cells. It is known that growth of stem cells as spheroids support better maintenance of their properties (reviewed in Cesarz and Tamama, 2016). In search of conditions/reagents that can promote growth of lymphocytes as spheroids, we decided to try to use agents that are able to stimulate intercellular adhesion between lymphocytes. As the main mechanism of intercellular contacts between activated lymphocytes is mediated by LFA-1-ICAM-1 interaction (Zumwalde et al. 2013), so it was investigated what polyclonal stimulators are able to activate LFA-1. One possibility is to employ stimulators based on antibodies against CD3, one component of TCR complex, as it has been demonstrated that such antibodies induce LFA-1-mediated lymphocyte adhesion (Dustin et al. 1989; Van Kooyk et al. 1989) and cluster formation (Rudnicka W et al., 1992).

In the examples presented below, the inventor employed the same CD3/CD28 antibody bound to polysterene beads (Dynabeads, ThermoFisher) as were used to stimulate initial proliferation of cells. It was found that addition of CD3/CD28 antibody 5 days after the initiation of the immunization step increases growth of lymphocytes as spheroids. Importantly, the final product contains cells with significantly upregulated expression of CD27 and CCR7m and, according to FACS analysis, with a phenotype resembling the stem cell memory T lymphocytes described by Gattinoni et al. (2011).

A further surprising finding is that the addition of CD3/CD28 antibody 5 days after the initiation of the immunization step also improves the above-mentioned ALECSAT-1 and -2 protocols in respect of the quality of cells obtained.

So, in a first aspect the present invention relates to a method for preparation of a composition comprising activated human CD8+ and natural killer (NK) lymphocytes, comprising
1) isolating a sample of blood cells from a subject (preferably human), wherein the sample is enriched for lymphocytes;
2) culturing a fraction of the sample in the presence of at least one agent capable of activating T lymphocytes via binding to CD3 and/or CD28 thereby stimulating proliferation of CD4+ lymphocytes and increasing the CD4+/CD8+ ratio compared to the lymphocytes obtained from step 1;
3) contacting the T lymphocytes with an agent that induces expression of cancer/testis antigens followed by a period of culture that results in said expression of cancer/testis antigens;
4) separating the proliferating T lymphocytes from the agent capable of activating T lymphocytes and mixing the cancer/testis antigen expressing lymphocytes obtained from step 3 with a second fraction of the sample from step 1; and
5) subsequently culturing the lymphocyte mixture from step 4 to stimulate proliferation of CD8+ and NK lymphocytes.

This process is also termed ALECSAT-3 herein.

In a second aspect, the present invention relates to a method for preparation of a composition comprising activated human CD8+ and natural killer (NK) lymphocytes, comprising
a) isolating a sample of blood cells from a subject, wherein the sample is enriched for lymphocytes;
b) culturing a fraction of the sample under conditions that stimulate proliferation of CD4⁺ lymphocytes and increase the CD4⁺/CD8⁺ ratio compared to the lymphocytes obtained from step a;
c) contacting the proliferating T lymphocytes with an agent that induces expression of cancer/testis antigens followed by a period of culture that results in said expression of cancer/testis antigens;
d) separating the cancer/testis antigen expressing T lymphocytes from the agent capable of activating T lymphocytes followed by mixing the cancer/testis antigen expressing lymphocytes with a second fraction of the sample from step a; and
e) subsequently culturing the lymphocyte mixture from step 4 to stimulate proliferation of CD8⁺ and NK lymphocytes,
wherein step e) comprises addition of an agent capable of activating T lymphocytes via binding to CD3 and/or CD28.

In a third aspect, the present invention relates to a method for treatment of cancer in a patient (preferably human), comprising administering a composition of cells prepared according to the method of the first or second aspect of the invention and embodiments thereof disclosed herein.

In fourth aspect, the present invention relates to a composition of cells prepared according to the method of the first or second aspect of the invention and embodiments thereof disclosed herein, for use in therapy.

Finally, in a fifth aspect, the present invention relates to a composition of cells prepared according to the method of the first or second aspect of the invention and embodiments thereof disclosed herein for use in a method according to the third aspect of the invention and embodiments thereof disclosed herein.

### LEGENDS TO THE FIGURE

FIG. 1: Bar graphs showing expression of MAGE antigens by lymphocytes co-cultured with dendritic cells and subsequently treated with 5 Aza-CdR.

For each MAGE antigen (MAGE A1, A3, A4, A6, A10, and A12, designated as M1, M3, M4, M6, M10, and M12), the bars represent MAGE expression:

White bar: after 4 days of co-culture and treatment with 5 Aza-CdR.

Obliquely hatched bar: after 5 days of co-culture and treatment with 5 Aza-CdR.

Horizontally hatched bar: after 6 days and treatment with 5 Aza-CdR.

The two panels represent measurements obtained from two different lymphocyte cultures.

FIG. 2. Outline of an embodiment of the inventive protocol of generation of cytotoxic lymphocytes; see example 1 for details of a practical implementation

FIG. 3. Bar graphs showing expression of MAGE antigens after treatment of activated lymphocytes from two donors (23-21 24-21) with 5-aza-2′-deoxycytidine (5-Aza-CdR).

FIG. 4. Bar graphs showing phenotype of the 5-Aza-CdR-treated cells.

A: Per donor measurements.

B: Average for all dononrs.

FIG. 5. Picture showing lymphocytes in culture according to the present invention. The lymphocytes grow typically as spheroids by the end of cultivation period.

FIG. 6. Bar graph showing the effect of addition of CD3/CD28 Dynabeads on the expression of CD27, CD62L and CCR7 on lymphocytes by the end of immunization/expansion steps.

White bar: no addition of CD3/CD28 Dynabeads.

Cross-hatched bar: addition of CD3/CD28 Dynabeads.

FIG. 7. Double logarithmic plots of flowcytometric data showing the effect of addition of CD3/CD28 Dynabeads on the expression of CCR7 and CD45RA on lymphocytes in the end of immunization/expansion steps.

FIG. 8. Double logarithmic plots of flowcytometric data showing the effect of addition of CD3/CD28 Dynabeads on the expression of CCR7 and CD45RA on lymphocytes of glioblastoma patients in the end of immunization/expansion steps.

FIG. 9. Line graphs showing cytolytic activity of the generated effector cells against T47D and MDA-MB-231 breast cancer cell lines.

FIG. 10. Bar graph showing cytolytic activity of the generated effector cells against four breast cancer cell lines.

FIG. 11. Outline of the protocol of generation of ALECSAT-1 (A) and ALECSAT-1/3 (B); see example 4 for details of a practical implementation.

FIG. 12. Outline of the protocol of generation of ALECSAT-2 (A) and ALECSAT-2/3 (B); see example 4 for details of a practical implementation.

FIG. 13. Bar graphs showing the effect of addition of CD3/CD28 Dynabeads on (A) lymphocyte expansion and (B) on the expression of CD27, CCR7 and TIGIT on CD8+ T lymphocytes in the end of immunization/expansion steps prepared according to protocol ALECSAT-1.

FIG. 14. Bar graphs showing the effect of addition of CD3/CD28 Dynabeads on (A) lymphocyte expansion and (B) on the expression of CD27, CCR7 and TIGIT on CD8+ T lymphocytes in the end of immunization/expansion steps prepared according to protocol ALECSAT-2.

### DETAILED DISCLOSURE OF THE INVENTION

### Definitions

“Cancer/testis antigens” (CTAs) is a group of antigens, which are expressed by a broad spectrum of cancers. The group includes such antigens as MAGE (including MAGE-1, MAGE-2, and MAGE-3), BAGE, GAGE, NY-ESO-1 and BORIS, which are all cancer-associated antigens that can be safely targeted, since they are not normally expressed in healthy cells in vital tissues. Previously it has been demonstrated that the expression by cancer cells of CTAs is a consequence of genome-wide de-methylation (including promoter de-methylation at CpG regions), which occurs in many cancers.

“Mononuclear cells” (also term “peripheral blood mononuclear cells”, abbreviated PBMC) denotes any cells of peripheral blood that have a rounded nucleus. The two main types of mononuclear cells are lymphocytes and monocytes, of which the latter have the ability to differentiate into macrophages and dendritic cells.

“Mature dendritic cells” (mature DCs) are in the present context dendritic cells that are obtainable by culturing monocytes under conditions described herein in the comparative part of Example 1 and which—in contrast to immature dendritic cells—exhibit a high potential for T-cell activation. These mature dendritic cells, which are obtained by plating and culturing adhering monocytes, subsequently treating with IL-4 (and/or IL-13) and GM-CSF to differentiate the monocytes into immature DCs and thereafter treating the immature DCs with TNF-alpha, IL-1beta, IL-6, and prostaglandin E2, are not loaded with antigen as would be the case for mature DCs isolated from lymphoid tissue.

“CD4⁺ lymphocytes” or “CD4⁺ cells” (the terms are used interchangeably herein) refer to lymphocytes of the T-helper subset. Among their functions are stimulation of B-cells and they also play an important role in the activation of CD8⁺ lymphocytes.

“CD8⁺ lymphocytes” or “CD8⁺ cells” or “cytotoxic T cells” (the terms are used interchangeably herein) refer to antigen specific lymphocytes that are capable of recognizing and killing cells that display MCH class I restricted T-cell epitopes.

“Natural killer cells” or “NK cells” or “NK lymphocytes” are antigen unspecific lymphocytes, which form part of the fast-reacting innate immune system, and which, as is the case of cytotoxic T cells, have the ability to kill cells. This occurs as part of recognition of stress-induced proteins characteristic for cancer cells. NK cells have a preferential ability to target cells that do not express MHC class I molecules.

The expression “increasing the CD4+/CD8+ ratio” is in the present context meant to indicate that a lymphocyte population that has been co-cultured with mature DCs as taught herein provides for a preferential expansion of the CD4⁺ subset of lymphocytes. It has been demonstrated that such co-culture, which forms part of the technology disclosed in WO 2008/081035, provides for a significant increase in CD4⁺ cells compared to CD8⁺ cells.

“An agent that induces expression of cancer/testis antigens” denotes a substance or composition, which is able to produce—in a treated cell—an effect corresponding to what has been observed in many cancers, namely that CTAs are expressed due to genome-wide changes. Typically, substances that can cause DNA to de-methylate are useful; good examples are 5-aza-2′-deoxycytidine, 5-azacytldine, 5-fluoro-2′-deoxycytidine, guadecitabine, and zebularine. Of these, the preferred de-methylation agent is 5-aza-2′-deoxycytidine (also termed 5-Aza-CdR or simply AzaC herein), which is a cytidine analogue that acts as a nucleic acid synthesis inhibitor. This substance under the name decitabine (marketed under the tradename DACOGEN®) acts via inhibition of DNA methyltransferase. As a viable alternative to use of de-methylating agent can be mentioned use of agents that induce the CTAs by means of histone acetylation-an example of such an agent is the histone deacetylase inhibitor trichostatin A.

An “agent capable of activating T lymphocytes via binding to CD3 and/or CD28” is a substance or composition of matter, which is capable of binding to CD3 (cluster of differentiation 3) and/or CD28 (cluster differentiation factor 28) with the effect that the T lymphocytes are activated. CD28 is naturally the receptor for CD80 and CD86, meaning that soluble versions of these molecules could function as T-cell activators. Normally, antibodies binding to CD3 and/or CD28 are used for the purpose of activating T-cells, and also bispecific antibodies that bind both molecules are available commercially. Herein, in the examples, are used hyperparagmagnetic bead-coupled monoclonal antibodies.

The expressions “immunization step” and “in vitro immunization” and “expansion step” generally relate to the step of co-culturing the lymphocyte mixture, where the CD4⁺ enriched lymphocytes immunize a fraction of the original lymphocytes.

### SPECIFIC EMBODIMENTS OF THE INVENTION

### Embodiments of the 1^{st} Aspect of the Invention

Step 1 of the method is carried out as generally known in the art: a blood sample is fractionated by methods known per se and a fraction of the blood sample is prepared, which predominantly contains lymphocytes. For instance, peripheral mononuclear cells (PBMCs) can be isolated by simple density gradient technologies followed by an appropriate adsorption technology for separating lymphocytes from other PBMCs, cf. example 1.

In step 2, culture of a portion of the isolated lymphocytes is carried out under circumstances that sustain their growth and facilitate their activation, and as an important feature, the agent that binds CD3 and/or CD28 (preferably both) is admixed with the cells at the onset of the cultivation step. The duration of this cultivation step is between 2 and 5 days, preferably about 3 days as demonstrated in the examples. In other words, cultivation of the T lymphocytes is carried out for about 48, about 49, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 101, about 102, about 103, about 104, about 105, about 106, about 107, about 108, about 109, about 110, about 111, about 112, about 113, about 114, about 115, about 116, about 117, about 118, about 119, or about 120 hours.

Step 3 is carried out at previously described in WO 2020/208054. During this period—and after step 1—the second fraction of the sample from step 1 is kept frozen between steps 1 and 2.

In step 3, the agent that induces expression of cancer/testis antigens is hence typically a DNA de-methylating agent or a histone acetylating agent. The DNA de-methylating agent is preferably selected from 5-aza-2′-deoxycytidine (5 Aza-CdR, which is the most preferred agent for this purpose), 5-azacytldine, 5-fluoro-2′-deoxycytidine, guadecitabine, and zebularine, and wherein the histone acetylating agent is Trichostatin A or a depsipeptide. Step 3 usually has a duration of about 2 days, i.e. between 36 and 60 hours.

During all steps of lymphocyte culture, IL-2 or another agent, such as IL-15, IL-7, and IL-21 (which all stimulate proliferation of lymphocytes), is added during the course of culture of lymphocytes.

In a preferred embodiment, step 5 (the “immunization step”) also entails addition of an agent capable of activating T lymphocytes via binding to CD3 and/or CD28, preferably the same type of agent as used in step 2; however, it is not of paramount importance that the agent is identical in the 2 steps—the exact choice will be governed by convenience.

The addition of the agent in step 5 usually takes place on day 3-7 after initiation of step 5, preferably after about 5 days. From this point on, cultivation is carried out until the human CD8+ and natural killer cell composition can be isolated/recovered from the culture mixture.

All in all, the method of the first aspect has a duration of at most 20 days, but with a duration of at most 16 days being preferred (step 1+2≈3 days, step 3≈2 days, and steps 4+5≈11 days). This is a significant shorter time for provision of the activated T cells disclosed in the past.

The agent capable of activating T lymphocytes via binding to CD3 and/or CD28 normally comprises antibodies, antibody fragments or antibody analogues, which bind CD3. However, other binding specific molecules are envisioned for this purpose—for instance, nucleic acid or peptide aptamers can be employed, as can molecular imprinted polymers prepared by using CD3 as a template would have the same functionality as would any properly selected binding partner for CD3. Likewise, the agent capable of activating T lymphocytes via binding to CD3 and/or CD28 normally comprises antibodies, antibody fragments or antibody analogues, which bind CD28 but here soluble versions of CD80 or CD86 constitute a useful alternative as does aptamers and molecular imprinted polymers.

In a preferred embodiment, the agent capable of activating T lymphocytes via binding to CD3 and/or CD28 comprises antibodies, antibody fragments or antibody analogues which bind CD3 and comprises antibodies, antibody fragments or antibody analogues which bind CD28. Also here, alternative agents as those described above (aptamers, molecular imprinted polymers and soluble receptors) can be used, but it is also possible to utilise antibody analogues that are multispecific (e.g. bispecific) for CD3 and CD28.

As shown in the examples, excellent results are obtained when the antibodies, antibody fragments or antibody analogues are linked to a solid or semi-solid phase. The use of the agents capable of activating T lymphocytes via binding to CD3 and/or CD28 coupled to such a solid or semi-solid phase is hence a preferred embodiment of the 1^{st} aspect of the invention. The solid or semi-solid phase is in useful embodiments constituted by separable beads, such as paramagnetic or superparamagnetic beads. However, it is also within the scope of the present invention to utilise polymers such as dextran, PEG, and other polymers for coupling.

### Embodiments of the Second Aspect of the Invention

As shown in Example 4, it surprisingly turns out that the prior art processes ALECSAT-1 and ALECSAT-2 can both be improved by the addition of CD3/CD28 antibody 5 days after the initiation of the immunization step, exactly as is the case in respect of the method of the first aspect of the invention, see above.

This finding hence evidences that the outcome of methods of the ALECSAT technology type can generally be improved by utilisation of the interaction with CD3 and/or CD28 during the immunization/expansion step. Hence, according to the 2^{nd} aspect of the invention, a method is provided for preparation of a composition comprising activated human CD8+and natural killer (NK) lymphocytes, comprising
a) isolating a sample of blood cells from a subject, wherein the sample is enriched for lymphocytes;
b) culturing a fraction of the sample under conditions that stimulate proliferation of CD4⁺ lymphocytes and increase the CD4⁺/CD8⁺ ratio compared to the lymphocytes obtained from step a;
c) contacting the proliferating T lymphocytes with an agent that induces expression of cancer/testis antigens followed by a period of culture that results in said expression of cancer/testis antigens;
d) separating the cancer/testis antigen expressing T lymphocytes from the agent capable of activating T lymphocytes followed by mixing the cancer/testis antigen expressing lymphocytes with a second fraction of the sample from step a; and
e) subsequently culturing the lymphocyte mixture from step 4 to stimulate proliferation of CD8⁺ and NK lymphocytes,
wherein step e) comprises addition of an agent capable of activating T lymphocytes via binding to CD3 and/or CD28.

In other words, step b can for instance be carried out using the prior art ALECSAT-1 or ALECSAT-2 production technologies where autologous mature dendritic cells are co-cultured with the lymphocytes prior to the induction of expression of cancer/testis antigens, and where autologous dendritic cells are used as co-culture cells before or within the immunization step. Hence in respect of step b, all technical details pertaining to preparation of mature dendritic cells and their use in the methods disclosed in WO 2008/081035 and WO 2020/208054 can be applied mutatis mutandis to the process of the second aspect of the invention.

Generally, in embodiments of this aspect, the agent capable of activating T lymphocytes via binding to CD3 and/or CD28 is employed and has the characteristics already described above in the discussion of the first aspect of the invention and the embodiments thereof.

Hence. the agent capable of activating T lymphocytes via binding to CD3 and/or CD28 is typically added 3-7 days after initiation of step 5, preferably after about 5 days; the the agent capable of activating T lymphocytes via binding to CD3 and/or CD28 preferably comprises antibodies, antibody fragments or antibody analogues which bind CD3; the agent capable of activating T lymphocytes via binding to CD3 and/or CD28 preferably comprises antibodies, antibody fragments or antibody analogues which bind CD28; the agent capable of activating T lymphocytes via binding to CD3 and/or CD28 preferably comprises antibodies, antibody fragments or antibody analogues which bind CD3 and comprises antibodies, antibody fragments or antibody analogues which bind CD28; the agent capable of activating T lymphocytes via binding to CD3 and/or CD28 are preferably linked to a solid or semi-solid phase, or to a polymer, such as dextran; and the agent capable of activating T lymphocytes via binding to CD3 and/or CD28 are linked to the solid or semi-solid phase, which is constituted by separable beads, such as paramagnetic or superparamagnetic beads.

Also, the method of the 2^{nd} aspect and the above described embodiments thereof is typically followed by isolation/recovery of the activated CD8+ and NK lymphocytes.

As mentioned, the conditions in step b can e.g. be those that characterize the prior art ALECSAT-2 or 3, technologies, i.e. entail co-culture with mature dendritic cells prepared from the sample in step a.

Likewise, depending on whether the agent capable of activating T lymphocytes via binding to CD3 and/or CD28 is employed in the ALECSAT-1 or -2 method, any or all of steps c, d or e comprises addition of mature dendritic cells prepared from the sample in step a. Again, in respect of these steps, all technical details pertaining to preparation of mature dendritic cells and their use in the methods disclosed in WO 2008/081035 and WO 2020/208054 can be applied mutatis mutandis to the process of the second aspect of the invention.

Consequently, the contents of WO 2008/081035 and WO 2020/208054 are incorporated by reference herein.

### Modifications of ALECSAT-1 or -2 Processes

In specific embodiments, the second aspect of the invention include all steps of the prior art ALECSAT-1 or ALECSAT-2 methods:

Thus, the agent capable of activating T lymphocytes via binding to CD3 and/or CD28 is preferably used in one of the prior art ALECSAT-1 or -2 methods, that is, in a method for preparation of a composition comprising activated human CD8⁺ and natural killer (NK) lymphocytes, where the method comprises i) isolating mononuclear cells from a blood sample from a human donor and separating the PBMCs into a fraction enriched for monocytes and a fraction enriched for lymphocytes; ii) culturing a portion of the monocyte-enriched fraction under conditions that facilitate maturation of dendritic cells; iii) subsequently mixing a first portion of the mature dendritic cells obtained in step ii with a first portion of the lymphocyte fraction obtained in step i; iv) co-culturing the mixed cells obtained in step iii to stimulate proliferation of CD4+ lymphocytes, thereby increasing the CD4⁺/CD8⁺ ratio compared to the lymphocytes obtained from step i; v) isolating proliferating lymphocytes from the co-cultured cells of step iv, and subsequently contacting them with an agent that induces expression of cancer/testis antigens followed by a period of culture that results in said expression of cancer/testis antigens; vi) mixing the cancer/testis antigen expressing lymphocytes obtained in step v with a second portion of the fraction enriched for lymphocytes in step i; and vii) subsequently culturing the lymphocyte mixture from step vi to stimulate proliferation of CD8+ and NK lymphocytes. This is the generic ALECSAT-1 process, which can further comprise a late addition of the mature dendritic cells obtained from step ii, namely after the co-culture of the cancer/testis antigen expressing cells. In contrast, the generic ALECSAT-2 process comprises that a second portion of mature dendritic cells obtained from step ii are added to the proliferating lymphocytes in any of steps v-vii and at the latest 6 days after step vi.

Steps i-ii together last about 6 days, steps iii-iv together last about 7 days, and step v lasts 2 days. In the ALECSAT-2 process steps 6-8 together last about 11 days, where it is preferred that the second portion of mature dendritic cells is added 13-17 days after commencement of step ii, preferably 15-17 days after commencement of step ii.

Step i consists of a step of separation of monocytes from lymphocytes after provision of a sample of PBMCs. After this separation, both the lymphocyte fraction and the monocyte fractions are divided into at least 2 portions each. Since cells from the lymphocyte fraction are not entering the process described above until after step ii, and since further cells from the lymphocyte fraction are not entering the process until after step v, the at least 2 portions of the fraction enriched for lymphocytes is frozen, one between steps i and ii, and another between steps i and vi. Likewise, the second portion of the mature dendritic cells is kept frozen between step ii and the addition of this portion in step v or step vi (and/or later after the culture of the cancer/testis antigen expressing lymphocytes). Under normal circumstances, the first portion of the mature DCs is used directly after step 1, i.e. without being frozen.

Step ii is essentially carried out according to known methods for preparing mature DCs from monocytes in culture; these known methods include addition, during the course of culture, of granulocyte-macrophage colony stimulating factor (GM-CSF) and Interleukin 4 (IL-4) (and/or Interleukin 13) to obtain immature DCs, followed by addition of TNFα to obtain the mature DCs. Additionally, Interleukin 1β (IL-1β), Interleukin 6 (IL-6), and prostaglandin E2 (PGE2) can advantageously be added in the phase of preparing the mature DCs.

Steps iv-vii are generally carried out as disclosed in WO 2008/081035 unless when applying the early addition of mature DC feeder cells in steps v-vii, when employing the ALECSAT-2 approach. Hence, any generic disclosure relating to these steps also apply to the process described in this section.

The ALECSAT-2 process can also be defined by the steps of I) contacting a first composition of human cells comprising proliferating CD4⁺ lymphocytes with an agent that induces expression of cancer/testis antigens followed by a culturing period that results in said expression of cancer/testis antigens by cells in the first composition; and II) adding a second composition of human cells comprising unstimulated peripheral blood lymphocytes to the first composition of cells and culturing the combined compositions of cells to stimulate proliferation of CD8⁺ and NK lymphocytes; wherein a third composition of human cells comprising mature dendritic cells is added in step I or II and at the latest 6 days after initiation of step III, and wherein the first, second and third compositions of human cells are isogeneic. Also in this process, the agent capable of activating T lymphocytes via binding to CD3 and/or CD28 can be employed in step II as generally described above.

Thus, this version of the ALECSAT-2 method sets out at the point where a culture of proliferating lymphocytes (the first composition) has been established. Preferably the first composition is enriched for CD4⁺ lymphocytes relative to CD8⁺ lymphocytes, meaning that the ratio CD4⁺ lymphocytes/CD8⁺ lymphocytes is significantly increased compared to that found in normal blood (where the ratio is about 2).

The remaining steps in this simplified ALECSAT-2 process correspond to steps v-vii described above and any of the characteristics of these step can be applied mutatis mutandis. This is for instance the case with any disclosure that relates to the characteristics of the agent that induces expression of CTAs. As indicated, the 3 compositions of cells used in steps I and II are isogeneic, i.e. the cells are derived from cells of the same person or are for other reasons cells having the same genome.

Finally, the ALECSAT-2 can also set out after a composition comprising CTA expressing CD4⁺ lymphocytes has been provided. The ALECSAT-2 method thus comprises mixing a first composition of human cells comprising cancer/testis antigen expressing CD4+ lymphocytes with a second composition of human cells comprising unstimulated peripheral blood lymphocytes and culturing the combined compositions of cells to stimulate proliferation of CD8+ and NK lymphocytes; wherein a third composition of human cells comprising mature dendritic cells is added to the combined compositions at the latest 6 days after mixing the first and second composition and wherein the first, second and third compositions of cells are isogeneic. Again, in this version of the ALECSAT-2 process, the agent capable of activating T lymphocytes via binding to CD3 and/or CD28 can be employed in as generally described above

In the above-described ALECSAT-1 and-2 methods, where the agent capable of activating T lymphocytes via binding to CD3 and/or CD28 can be employed, it is preferred that the mature dendritic cells are unloaded with antigen and that they are non-irradiated. Normally, irradiation of the feeder cells is employed in order to prevent them from proliferating, but the mature dendritic cells used in the ALECSAT-1 and -2 methods do not proliferate or at least exhibit an acceptably low degree of proliferation. Further, use of peptide loaded dendritic cells has been used to stimulate CD8⁺ cells, but the ALECSAT methods have been shown to stimulate proliferation of CD8⁺ cells as well.

In all culturing steps, it has been found that IL-2 advantageously can be applied, cf. the examples.

As already described above, the early addition of the mature DCs in the ALECSAT-2 method as feeder cells is at the latest 6 days after instigation of the co-culture of the CTA expressing CD4⁺ lymphocytes and the non-stimulated lymphocytes; typically this is at the latest 5 days, at the latest 4 days, at the latest 3 days, and at the latest 2 days. Cf. above under the 1^{st} aspect of the invention for details concerning the timing for this early addition. However, it is preferred that the addition of the feeder cells is at the latest or exactly 0, 1 or 2 days after instigation of the co-culture of the CTA expressing CD4⁺ lymphocytes and the non-stimulated lymphocytes.

The last culture step is typically followed by isolation/recovery of the activated CD8+ and NK lymphocytes. These are then typically subsequently preserved for later use in therapy or they are used directly in the patient from which the cells are derived.

### Embodiments of the Third Aspect of the Invention

This method is in essence detailed in WO 2020/208054, with the sole difference being that the cells administered to the patient are produced by the method of the first or second aspect of the present invention. Hence, in this method, it is preferred that the cells administered are the patient's autologous cells, which have been modified by means of the method of the first or second aspect of the invention as well as any embodiments thereof disclosed herein. Typically, the patient receives at least or exactly 2, at least or exactly 3, or at least of exactly 4 administrations of the modified cells.

Administration of the cells is conveniently via the parenteral route, such as the intraveneous, intraarterial route, intratumoral route, and intralymphatic route.

Further the cancer is selected from the group consisting of carcinoma, adenocarcinoma, sarcoma (including liposarcoma, fibrosarcoma, chondrosarcoma, osteosarcoma, leiomyosarcoma, rhabdomyosarcoma), glioma (in particular glioblastoma), neuroblastoma, medullablastoma, malignant melanoma, neurofibrosarcoma, choriocarcinoma, myeloma, and leukemia.

In interesting embodiments of the third aspect of the invention, the treatment is combined with administration of an anticancer drug (i.e. a co-treatment), in particular with an immune checkpoint inhibitor drug (also termed simply a checkpoint inhibitor). Particularly preferred checkpoint inhibitor drugs in this context are inhibitors of PD-1 and/or PD-L1.

Hence, in a particular interesting embodiment, the method of the 3 aspect comprises a combination with anticancer treatment, where an effective amount of a PD-1 inhibitor or a PD-L1 inhibitor is administered. The co-treatment man take place prior to and/or concurrent with and/or after the treatment with the cells prepared according to the present invention.

The PD-1 and PD-L1 inhibitors can in this interesting embodiment be any of the following approved or experimental inhibitors:

### Approved PD-1 Inhibitors

Pembrolizumab (formerly MK-3475 or lambrolizumab, Keytruda), approved for the treatment of melanoma, metastatic non-small cell lung cancer and head and neck squamous cell carcinoma.

Nivolumab (Opdivo), approved for the treatment of melanoma, squamous cell lung cancer, renal cell carcinoma, and Hodgkin's lymphoma.

Cemiplimab (Libtayo), approved for the treatment of cutaneous squamous cell carcinoma (CSCC) or locally advanced CSCC who are not candidates for curative surgery or curative radiation.

Dostarlimab (Jemperli), approved for treatment of mismatch repair deficient (dMMR) recurrent or advanced endometrial cancer by the FDA in April of 2021.[13] On Aug. 17, 2021, the FDA granted accelerated approval for the treatment of mismatch repair deficient (dMMR) recurrent or advanced solid tumours.

### PD-1 Inhibitors Currently Being Investigated

JTX-4014 by Jounce Therapeutics, which is currently undergoing a Phase I clinical trial;

Spartalizumab (PDR001) developed to treat both solid tumours and lymphomas;

Camrelizumab (SHR1210), which has been conditionally approved for treatment of Hodgkin's lymphoma.

Sintilimab (IBI308) developed to treat non-small cell lung cancer (NSCLC).

Tislelizumab (BGB-A317), which is developed for treatment of solid tumors and hematologic cancers.

Toripalimab (JS 001).

INCMGA00012 (MGA012).

AMP-224.

AMP-514 (MEDI0680).

### Approved PD-L1 Inhibitors

Atezolizumab (Tecentriq), which is approved for treatment of urothelial carcinoma and non-small cell lung cancer.

Avelumab (Bavencio), which is approved for the treatment of metastatic merkel-cell carcinoma

Durvalumab (Imfinzi), which is approved for the treatment of urothelial carcinoma and unresectable non-small cell lung cancer after chemoradiation.

### PD-L1 Inhibitors Currently Being Investigated

KN035;

CK-301 for treatment of NSCLC;

AUNP12 (a 29-mer peptide);

CA-170 for treatment of mesothelioma patients; and

BMS-986189.

### The Fourth and Fifth Embodiments of the Invention

Generally, these embodiments relate to the compositions disclosed above for use as a medicament, and in particular in the method of the third aspect of the invention. Hence, all disclosure relating to the 3^{rd} aspect and the embodiments thereof applies mutatis mutandis to the fourth and fifth aspects.

### EXAMPLE 1

### Development of the Shortened Protocol of Generation of Cytotoxic Lymphocytes

### Existing Protocol for Comparison Purposes

Generation of dendritic cells, activation of lymphocytes, and treatment with DNA demethylating reagent were performed as described previously in WO 2008/081035. The procedure is for the sake completeness described below.

### Day 0

Buffy coats were obtained from the local Blood Bank. Upon arrival, blood (about 60 ml) was diluted with 60 ml of Ca and Mg free Dulbecco's Phosphate Buffered Saline (DPBS, Product No. BE17-512F, Cambrex, Belgium), and approximately 30 ml were layered on 15 ml of Lymphoprep® (Product No. 1053980, AXIS-SHIELD PoC AS, Norway) in four 50 ml tubes. After the first centrifugation at 200 G, 20 min, 20° C., 15-20 ml of the upper layer of plasma (so-called platelet rich plasma, PRP) were collected to a separate tube, and used for the preparation of serum. For this, CaCl₂ was added to a concentration of 25 mM, and after mixing, the plasma was transferred to a T225 flask (Nunc, Denmark), and placed in a CO₂-incubator. The flask was left in the CO₂-incubator until the next day. Centrifugation of tubes with Lymphoprep® was continued at 460 G, 20 min, 20° C. After termination of centrifugation, mononuclear cells were collected from the interface between Lymphoprep® and plasma to tubes with 25 ml of cold DPBS-EDTA (Cambrex) and washed three times with cold DPBS-EDTA by centrifugation, first at 300 G, then two times at 250 G, each time for 12 min at 4° C. After the last wash, cells were re-suspended in 30 ml of cold Ca and Mg free DPBS, and counted using a Moxi counter.

The concentration of monocytes was determined by gating the corresponding peaks of cells. Generation of dendritic cells (DCs) was performed in T225 tissue culture flasks pre-treated with 30 ml of 5% human AB serum in RPMI 1640. After removal of pre-treatment medium, 30 ml of a cell suspension containing 4-5×10⁷ monocytes in AIM-V medium were added. After 30 min of incubation at 37° C., non-adherent lymphocytes were collected, adherent monocytes rinsed twice with pre-warmed RPMI 1640 medium and further cultured in 30 ml of AIM-V medium. The collected lymphocytes were frozen in several aliquots of 25-30×10⁶ cells.

### Day 1

GM-CSF and IL-4 (both from Gentaur, Belgium, or CellGenix, Germany) were added to the flask with monocytes to final concentrations of 100 ng/ml and 25 ng/ml, respectively.

The T225 flask with the clotted plasma was transferred to a refrigerator and placed in an inclined position, with the clotted plasma down, and after 15-30 minutes, serum transferred to a 50 ml tube, and transferred to a −20° C. freezer.

### Day 2

A tube with the frozen serum was transferred to the refrigerator (4° C.).

### Day 3

GM-CSF and IL-4 (both from Gentaur, Belgium, or CellGenix, Germany) were added to the flask with monocytes to final concentrations of 100 ng/ml and 25 ng/ml, respectively.

Tubes with the thawed serum were centrifuged at 2000 G, 15 min, 20° C., and the supernatant was transferred to a new 50 ml tube. This serum (termed “later plasma-derived serum”) was stored at 4° C.

### Day 4

IL-1β, IL-6, TNF-α (all from Gentaur), and PGE2 (Sigma) were added to final concentrations of 10 ng/ml, 1000 IU/ml, 10 ng/ml and 0.2 μg/ml, respectively, in 10 ml of AIM-V medium.

### Day 6—Start Co-Culture of Dendritic Cells and Lymphocytes

Non-adherent dendritic cells were harvested, counted and used for the experiment. For this, the frozen lymphocytes were thawed, counted, and 2×10⁷ lymphocytes were mixed with 2×10⁶ of dendritic cells. After centrifugation, the mixture was re-suspended in 41 ml of lymphocyte medium consisting of AIM-V medium (Gibco, Invitrogen) and 2% autologous plasma derived serum, and placed in a T175 flask. The flask was placed to the side position. Two parallel cultures were set up.

### Day 7

IL-2 (Gentaur) was added in 2 ml of AIM-V medium at final concentration of 25 IU/ml.

### Day 10 (Day 4 of Co-Culture)

The first culture was harvested, counted, and treated with 5-aza-2′-deoxycytidine. The other culture was fed with 40 ml of fresh lymphocyte medium supplemented with IL-2 (50 U/ml), and the flask were placed to standard (flat) position. Treatment was performed in 40 ml of lymphocytes medium supplemented with IL-2 (150 IU/ml) and 10 μM 5-aza-2′-deoxycytidine (obtained from Sigma).

### Day 11 (Day 5 of Co-Culture)

40 ml of cell suspension was harvested, and the rest of culture was supplemented with 40 ml of fresh lymphocyte medium supplemented with IL-2 (50 U/ml). The harvested cells were counted and treated with 5-aza-2′-deoxycytidine as described above.

### Day 12 (Day 6 of Co-Culture)

The rest of the culture was harvested, counted, and treated with 5-aza-2′-deoxycytidine as described above.

The first treated culture was harvested, counted, and 2,5 Mio of cells was used to prepare the pellet. Pellet was kept at −80° C. until determination of the of MAGE antigens.

### Day 13

The second treated culture was harvested, counted, and 2.5 Mio cells were used to prepare the pellet. Pellet was kept at −80° C. until determination of the of MAGE antigens.

### Day 14

The third treated culture was harvested, counted, and 2,5 Mio of cells was used to prepare the pellet. Pellet was kept at −80° C. until determination of the of MAGE antigens.

Expression of MAGE antigens was determined as follows. The frozen pellets of cells were thawed, and total RNA was isolated from lymphocytes using the NucleoSpin RNA Plus kit (Macherey-Nagel), and reverse transcribed using SuperScript™ IV VILO™ Master Mix with ezDNase™ Enzyme (Invitrogen) and random hexamers and oligo(dT24) primers. cDNA was amplified in a LightCycler Nano (Roche) using the FastStart Essential DNA Green Master mix (Roche) and previously described primers and conditions (Weinert et al., 2009), except primers for MAGE-A4, provided by RealTimePrimers.com. The data were normalized to GAPDH expression. The list of primers is given in Table 1.

TABLE 1
Primer sequences for Q-PCR measurement
of MAGE genes.

| | | SEQ |
|---|---|---|
| Gene | Primer sequence (5′-3′) | ID NO: |
| MAGE A1, q s | AGTAGTAGGTTTCTGTTCTATTGGG | 1 |
| MAGE A1, q a | TACTTATTCCACTGCTGTTATTATCC | 2 |
| MAGE A3, q s | GCTGAGTGTGTTAGAGGTGTT | 3 |
| MAGE A3, q a | AGGGGTGGGTAGGAAATGT | 4 |
| MAGE A4, q s | TGTGATCTTCGGCAAAGCCT | 5 |
| MAGE A4, q a | TTTCCTGCACCCAATCTTGG | 6 |
| MAGE A6, q s | GCTGAGTGTGTTAGAGGTGTT | 7 |
| MAGE A6, q a | CAGGAGTGGGTAGGAAATGC | 8 |
| MAGE A10, q s | CCTGCCAGACAGTGAGTCTT | 9 |
| MAGE A10, q a | TGGGATCCACCTCCTTTACA | 10 |
| MAGE A12, q s | CTGAGTGTGTTGGAGGCATC | 11 |
| MAGE A12, q a | GGTGGGTAGGAAATGTGAGGT | 12 |
| GAPDH, q s | AGCTTGTCATCAATGGAAATCCC | 13 |
| GAPDH, q a | GTGAAGACGCCAGTGGACTC | 14 |

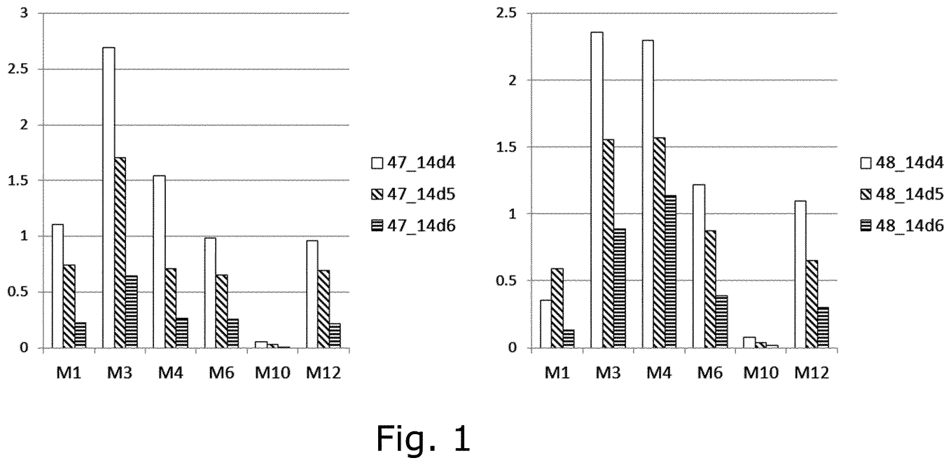

FIG. 1 shows the results of such determinations performed on two lymphocyte cultures. An increase in expression of MAGE antigens is apparent upon decreasing the time of co-culture from 6 to 4 days. Counting of small (non-activated) and large (activated) cells using a Coulter Counter indicates that only 30-40% of cells are activated at day 4, representing 17-30% of the initial number of cells (Table 2 below). These calculations show that despite the fact that significant CTA expression can be obtained by harvesting cells after only four days of co-culture, it will be very difficult if not impossible to generate enough amounts of 5-aza-2′-deoxycytidine-treated cells required for the next step in the procedure.

TABLE 2
Kinetics of activation of lymphocytes in co-culture with dendritic cells.

| | Concentration of cells (large/total), | | | Total mio. number of large | | |
|---|---|---|---|---|---|---|
| | mio./ml | | | cells per culture | | |
| Donor | Day 4 | Day 5 | Day 6 | Day 4 | Day 5 | Day 6 |
| 47_14 | 0.086/0.29 | 0.09/0.19 | 0.21/0.29 | 3.44 | 7.2 | 33.6 |
| 48_14 | 0.15/0.38 | 0.23/0.35 | 0.49/0.57 | 6.0 | 18.4 | 78.4 |

Starting number of cells per flask - 20 mio. in 41 ml.

For this reason, it was decided to investigate alternative ways of inducing polyclonal activation of T lymphocytes with predominant expansion of CD4⁺ cells and having much faster kinetics of activation. A CD3/CD28 antibody-based activation reagent that has fast kinetics of lymphocyte activation combined with preferential expansion of CD4⁺ cells (Thompson J A et al. (2003) was selected for this purpose.

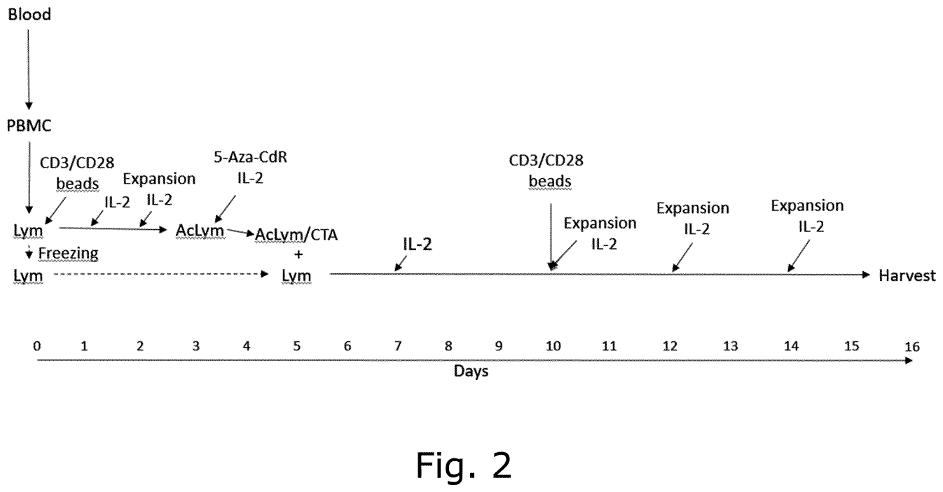

The new inventive protocol of lymphocyte activation is outlined in FIG. 2 and set forth in the following:

### Day 0

Lymphocytes were isolated from buffy coats as described above except that cells after the first adsorption were subjected to the second adsorption. Second adsorption was performed with 2×10⁷ cells in a T75 tissue culture flask pre-treated with 15 ml of 5% human AB serum in RPMI 1640. After 30 min of incubation at 37° C., non-adherent lymphocytes were collected and counted. After centrifugation of 1×10⁷ cells, the pellet was resuspended in 20 ml of lymphocyte medium consisting of AIM-V medium with addition of L-glutamine (2 mM) and 2% of autologous serum (prepared from the clotted plasma, see above). The cell suspension was placed in a T75 tissue culture flask (placed on its side). After addition of 100 μl of CD3/CD28 Dynabeads (Human T cell activator, ThermoFisher Scientific, cat. No 111.31D, 4×10⁷ beads/mL), the flask was transferred to a CO₂-incubator.

### Day 1

Interleukin 2 (IL-2) (Gentaur, Belgium) was added to the culture at the final concentration of 50 IU/ml.

### Day 2

20 ml of lymphocyte medium with addition of 25 IU/ml of IL-2 was added to the flask. The flask was positioned in the standard (flat) position.

### Day 3

The cell suspension was transferred to a 50 ml tube, and the cell concentration was determined. At this stage the culture contains only activated lymphocytes, and upon counting in a Moxi cell counter appear as a single peak of cells with an average size of 10-11 μm. 2×10⁷ cells (or all cells) were set for centrifugation. After centrifugation, the pellet was resuspended in 40 ml of lymphocyte medium, and after addition of IL-2 (150 IU/ml) and 10 μM 5-aza-2′-deoxycytidine (5-Aza-CdR, obtained from Sigma) cells were transferred to a T75 tissue culture flask.

### Day 5

5-Aza-CdR-treated cultures were harvested and centrifuged. After centrifugation, the pellet was resuspended in 3 ml of lymphocyte medium. The Dynabeads were depleted with the help of a magnet. The depletion procedure was repeated twice in order to ensure that no stimulatory beads are present at the start of the immunization period. After this, cells were counted, and 10×10⁶ cells were mixed with 10×10⁶ thawed lymphocytes, and after centrifugation, resuspended in 20 ml of lymphocyte medium and transferred to a T75 tissue culture flask, side position. 2.×10⁶ cells were set separately for centrifugation, and the resulting pellet was frozen and used later for determination of MAGE antigen expression by RT-PCR as described above.

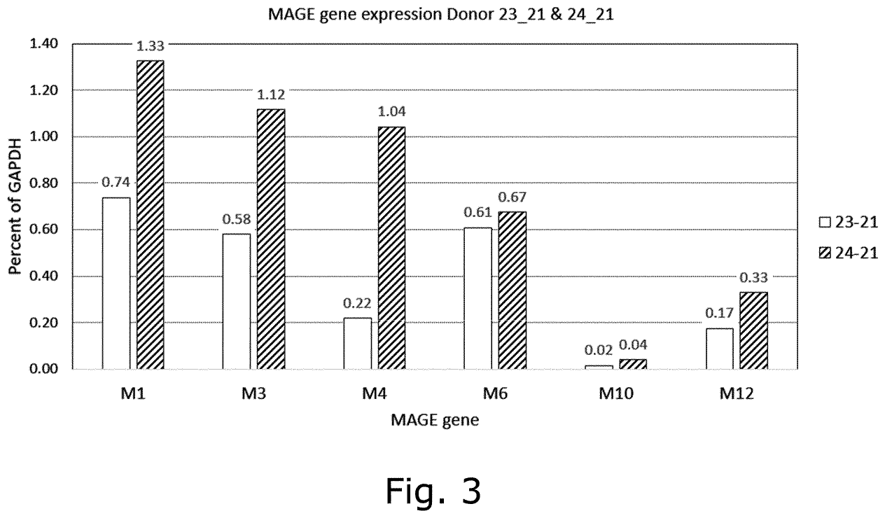

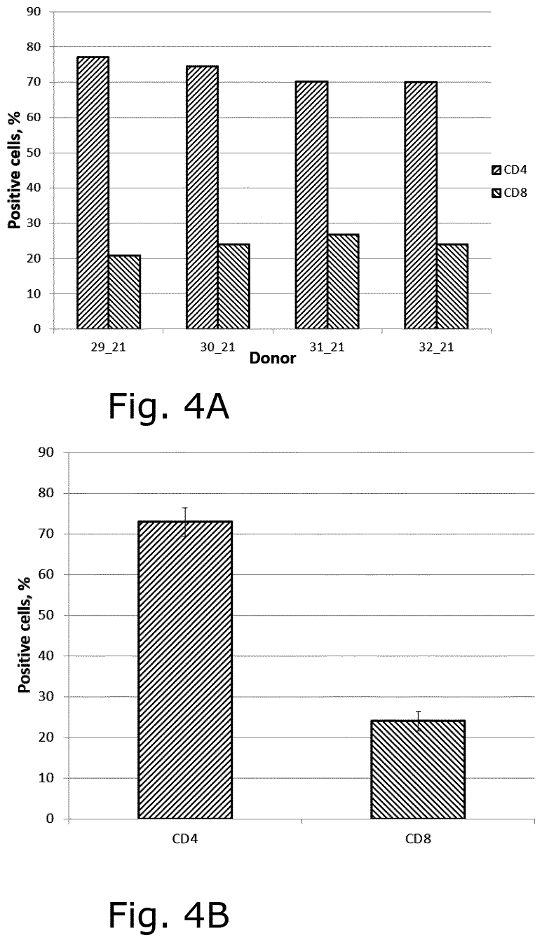

Robust and consistent expression of selected CT antigens of the MAGE family was achieved by treating cells with 10 μM 5-aza-CdR for 2 days in the presence of IL-2 (150 IU/ml) (see FIG. 3). Analysis of the 5-aza-CdR-treated cells for expression of surface markers indicates that CD4+ T lymphocytes are the predominant population of cells (FIG. 4).

### Day 7

20 ml of fresh lymphocyte medium supplemented with IL-2 (50 IU/ml) was added to the flask.

### Day 10

20 ml of cell suspension was transferred to a new T75 flask. 20 ml of fresh lymphocyte medium supplemented with IL-2 (50 U/ml) was added to each flask. 50 μl of CD3/CD28 Dynabeads (containing 2×10⁶ beads) were added to one of the flasks. Both flasks were placed to standard (flat) position.

Further cultivation (4-6 days) was performed by regular expanding and feeding of cultures with addition of fresh lymphocytes medium supplemented with IL-2 (final concentration of 25 IU/ml). At the end of the cultivation period (days 14-16), cultures were harvested, counted and used for analysis of phenotype and cytolytic activity.

### EXAMPLE 2

### Effect of Addition of CD3/CD28 Dynabeads 5 Days after Initiation of the Immunisation Step

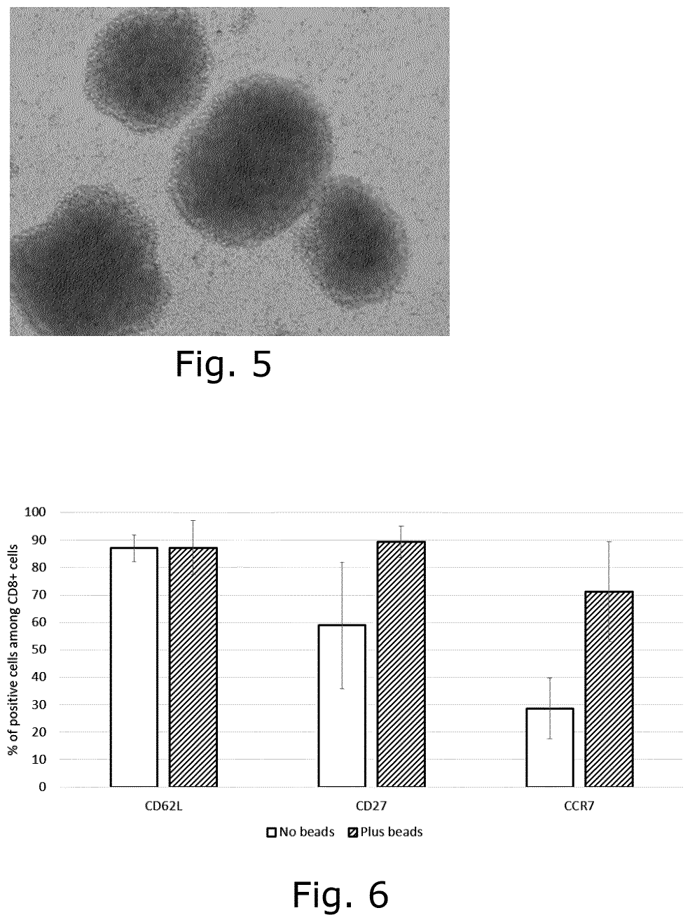

The expanded lymphocytes appear typically as spheroids under microscope investigation (FIG. 5). The phenotypes of the generated cells were determined by measurements of expression of surface markers by flow cytometric analyses. This was done by staining of cells with the directly conjugated antibodies, CD3-FITC, and CD62L-PE-Cy5 (Beckman Coulter, Sweden, A07746, IM2655), CD56-PE, CD4-FITC, CD8-PE, CD27-FITC, CCR7-FITC, CD45RA-PE-Cy5, and PD1-PE (BD Bioscience, 555516, 555346, 555635, 560986, 561271, 555490, and 560795). The recommended isotypic controls were used for the phenotyping of the cells. The cell samples were analysed using a Navios Flow Cytometer (Beckman Coulter) and the Kaluza analytical software version 1.3 (Beckman Coulter).

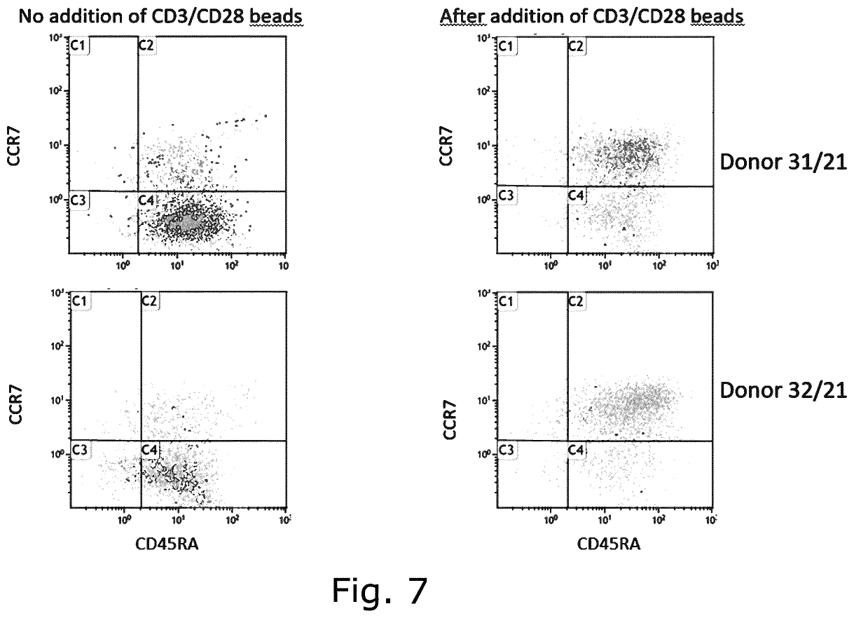

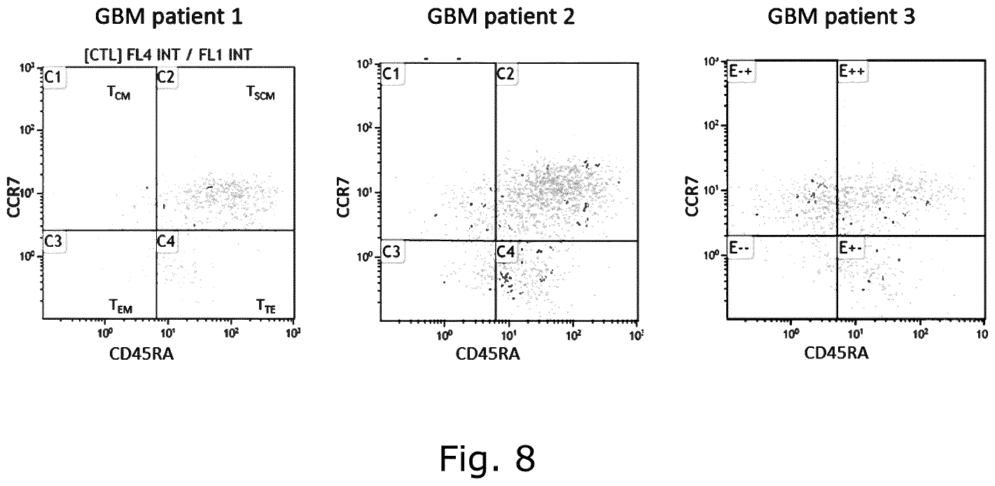

The effect of addition of CD3/CD28 Dynabeads on the phenotype of the expanded cells is shown in FIGS. 6 and 7. As can be seen, many parameters associated with the early differentiation state of CD8⁺ lymphocytes are significantly upregulated in cultures generated with addition of CD3/CD28 Dynabeads. The most dramatic increase is seen for the CCR7 chemokine receptor (FIG. 7). We have also performed preparation of cytotoxic cells according to the modified protocol from frozen lymphocytes of glioblastoma patients. As can be seen (FIG. 8), the new protocol with addition of CD3/CD28 Dynabeads generates cells with moderate to high levels of CCR7 expression 5 days after initiation of the immunization step.

It is of note that addition of CD3/CD28 beads 5 days after initiation of the immunization process was also described in Rosenblatt et al., 2010, but the authors did not monitor the expression of surface markers associated with the induction of early differentiation phenotype (CCR7, CD62L and CD27). Another group of authors (Rasmussen et al. 2010) employed CD3/CD28 Dynabeads for the expansion of antigen-prestimulated lymphocytes. The authors demonstrated that such expansion is capable of increasing the expression of some (CD62L), but not other (CCR7 and CD27) markers associated with early differentiation phenotype.

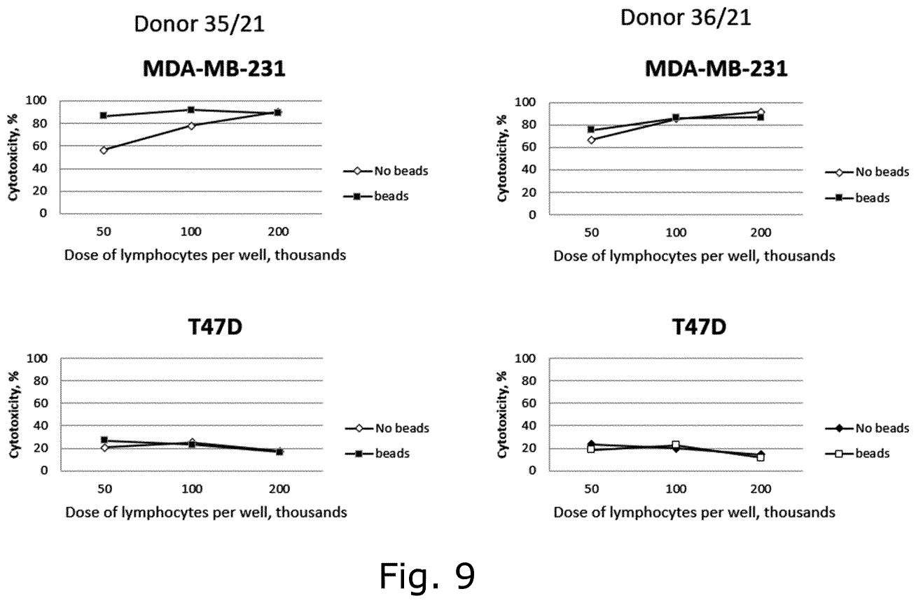

To determine the lytic activity of the effector lymphocytes, real-time cytotoxicity assays with the CELLigence system (ACEA Biosciences, San Diego, CA, USA) were employed. Two breast cancer cell lines were employed in most of the experiments: T47D (HLA-A2-) and MDA-MB-231 (HLA-A2+). Tumour cells were seeded at a density of 3×10⁴ cells per well in a total volume of 400 μL of RPMI 1640 medium with 10% FCS. After 2-4 h of initial incubation, three different doses (0.05×10⁶, 0.1×10⁶, and 0.2×10⁶) of lymphocytes were added in 200 μl of lymphocyte medium. Killing of tumour cells was associated with a decrease in cell impedance (measured as Normalized Cell Index) and was monitored every 15 minutes for 20-25 hours. The results of such determinations of the lytic activity of the effector cells from two donors are presented in FIG. 9. It can be clearly seen that the lytic activities of both culture 1 (without CD3/CD28 beads) and culture 2 (with CD3/CD28 beads) are similar. These results indicate that addition of CD3/CD28 Dynabeads increases the proportion of cells with early differentiation phenotype without negatively affecting their lytic activity.

### EXAMPLE 3

### Demonstration of TCR-Independent Lytic Activity of Cytotoxic T Lymphocytes

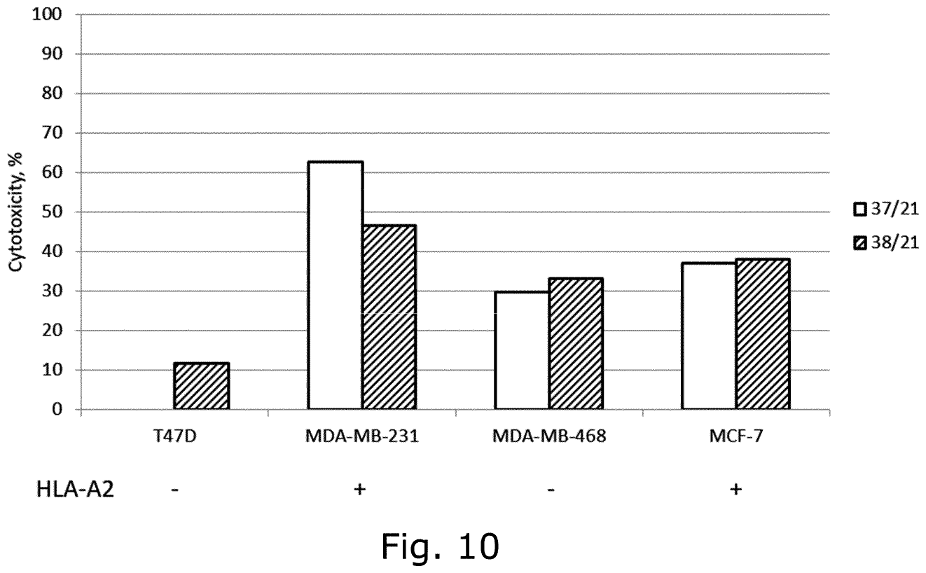

The present inventor has previously demonstrated that cytotoxic T lymphocytes generated against a 5-Aza-CdR-treated CD4-enriched population of lymphocytes are able to recognize antigen presenting cells loaded with peptide epitopes from two separate cancer testis antigens (NY-ESO-1 and MAGE-A10) in an ELISPOT assay, indicating that one of the components of specificity of the generated CTLs are indeed cancer testis antigens. On the other hand, the results presented in above Example 2 demonstrate the TCR-independent recognition of tumour cell line MDA-MB-231, indicating the presence of a TCR-independent component in the activity of the generated effector cells. To further investigate this matter, we have tested the lytic activity of the generated effector cells on a larger panel of tumour cell lines. The result of one such experiment is presented in FIG. 10. Here, four cell lines were employed: two were HLA-A2-positive, the other two were HLA-A2-negative. The generated effector cells were able to kill three cell lines with similar efficiency, while one cell line (T47D) was again much less sensitive to killing by generated effector cells. Higher sensitivity of MDA-MB-231 to lysis by the generated effector cells compared to T47D was observed for the effector cells generated totally for 16 donors, independent on the proportion of NK cells present in the preparation, and HLA-A2 phenotype of the effector cells, indicating that the lysis seen in this system is predominantly TCR-independent, and directed to structure(s) present on some tumour cell lines.

This observation resembles the TCR-independent recognition of tumour cells described by Braun et al. 2016, where it was demonstrated that CTLs showing antigen-specific recognition of normal cells loaded with the antigenic peptides, exert lysis of majority of tumour cell lines independent of HLA phenotype and the presence of the antigen. TCR-independent lysis of tumour cells described by Braun et al. 2016 was shown to be mediated by recognition of CD155 and/or CD112 by the molecule DNAM-1. Additional molecules involved in the lytic activity are CD45 and LFA-1 (which recognizes ICAM-1). The lytic activity also correlates with expression of CD62L. It is of note that the T47D breast cancer cell line resistant to TCR-independent lysis by T lymphocytes in the present experiments, does not express CD155, while the sensitive cell line MDA-MB-231 does (according to data presented by Zheng Q et al. 2019), further pointing to CD155 as a possible target for TCR-independent lysis seen with preparations of the effector cells. It is interesting that CD155 expression on tumour cells correlates with tumour progression, and CD155 is particular characteristic for triple negative breast cancers.

In summary, the above examples evidence the development of a new simplified protocol of generation of cytotoxic T lymphocytes cells with broad tumour specific lytic activity and with phenotypes of early differentiation lymphocytes.

### EXAMPLE 4

### Demonstration of Improvements of Quality of Cells ALECSAT Generated by the Previous Protocols by Addition of CD3/CD28 Beads 5 Days after Initiation of the Immunisation/Expansion Step

In Example 2 it is demonstrated that addition of CD3/CD28 antibodies 5 days after start of the immunisation process (i.e. the co-culture) significantly increases expression of markers associated with an early lymphocyte phenotype: CD27 and CCR7. This is specifically shown in cultures with the shortened preparation of CD4-enriched lymphocytes treated with a DNA demethylating agent.

In the current example it is further demonstrated that a similar increase in the same markers can be observed also in cultures where preparation of CD4-enriched lymphocytes treated with DNA demethylating agent were generated in a 15-days process described previously (cf. WO 2008/081035 and WO 2020/208054.

For the convenience of description of different variants of preparation of ALECSAT cells, we will employ the following names:

ALECSAT-1 (AL-1) designates ALECSAT cells prepared according to protocol described in WO 2008/081035.

ALECSAT-1/3 (AL-1/3) designates ALECSAT cells prepared according ALECSAT-1 protocol with addition of CD3/CD28 beads at day 20 of the process.

ALECSAT-2 (AL-2) designates ALECSAT cells prepared according to protocol described in the patent WO 2008/081035.

ALECSAT-2/3 (AL-2/3) designates ALECSAT cells prepared according ALECSAT-2 protocol with addition of CD3/CD28 beads at day 20 of the process.

ALECSAT-3 (AL-3) designates ALECSAT cells prepared during 16-day protocol described in the current patent in the example 2.

Generation of dendritic cells, CD4-enriched population of lymphocytes and treatment with DNA-demethylating agent were done as described in Example 1. Total duration of the process to this point is 15 days and consists of 6 days of preparation of dendritic cells, 7 days of co-cultivation of dendritic cells and lymphocytes, and 2 days of treatment with DNA-demethylation agent. Initiation of the immunisation step was done either in the absence (ALECSAT-1) or presence (ALECSAT-2) of dendritic cells. At day 20, each culture was split in two cultures, and to one of the cultures CD3/CD28 beads were added as described in Example 2. After additional cultivation for 6 days (day 26 of the total process), cultures were harvested and analysed by counting and by FACS analysis as described in Example 2 with the addition of measurement of expression of TIGIT (TIGIT-PE, 11-9500-41, Invitrogen).

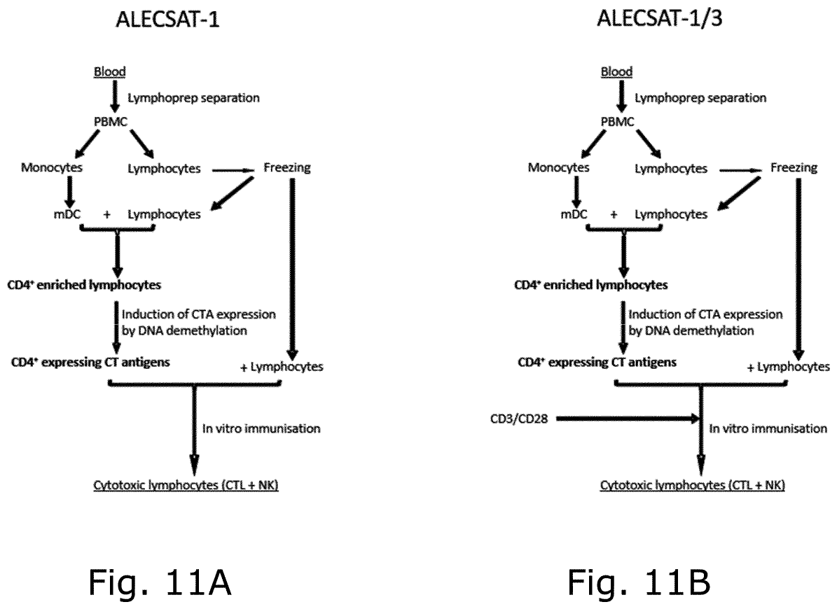

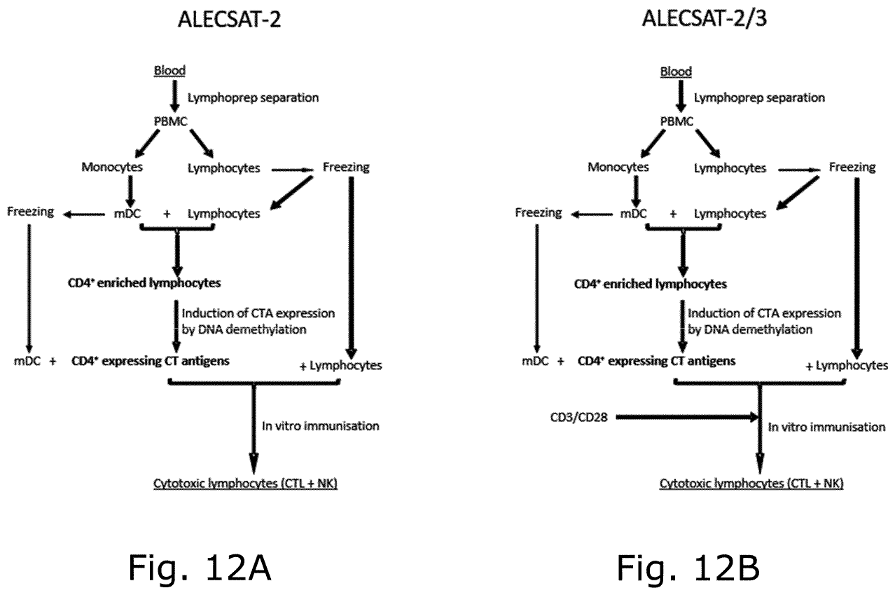

FIG. 11 demonstrates the outline of the protocols of generation of ALECSAT-1 (FIG. 11A) and ALECSAT-1/3 (FIG. 11B). FIG. 12 demonstrates the outline of the protocols of generation of ALECSAT-2 and ALECSAT-2/3 (FIGS. 12A and 12B, respectively).

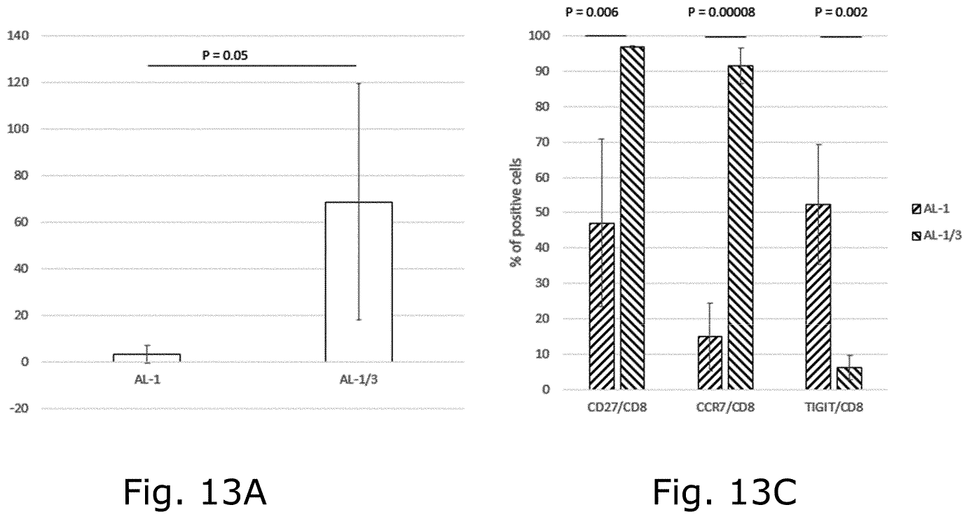

Addition of CD3/CD28 beads 5 days after initiation of the immunisation step to a preparation of ALECSAT-1 cells induces significant increase in the total number of the generated cells (FIG. 13A). There is also upregulation of expression of CD27 and CCR7 on CD8⁺ cells (FIG. 13B), similar to results seen with ALECSAT-3 preparation (FIG. 6 in Example 2). In addition, expression of the checkpoint TIGIT was decreased.

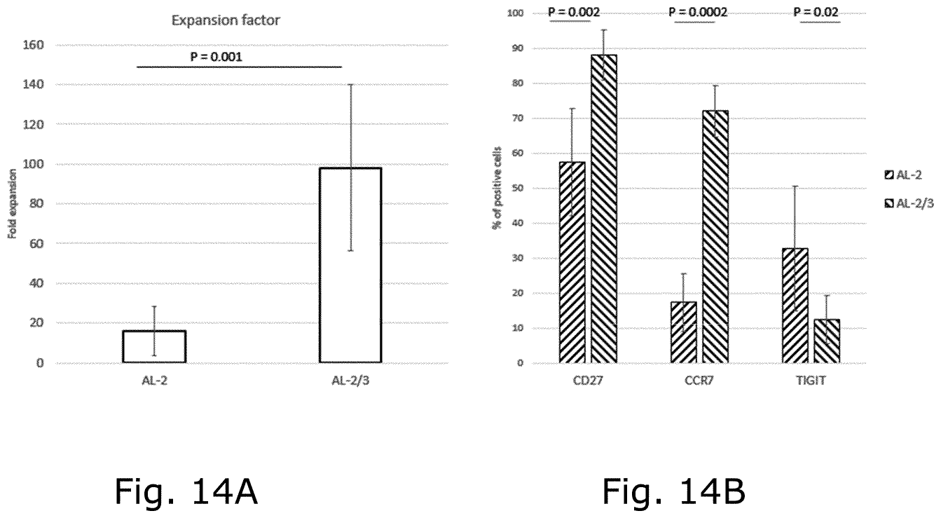

Similar results are seen upon addition of CD3/CD28 beads 5 days after initiation of the immunisation step to a preparation of ALECSAT-2 cells (FIG. 14, A and B).

In summary, the present example evidences significant improvements of the prior art ALECSAT-1 and ALECSAT-2 protocols by employment of one of the key elements of embodiments of ALECSAT-3 protocol, i.e. embodiments of the first aspect of the invention: the addition of CD3/CD28 microbeads 5 days after initiation of the immunisation step.

### LIST OF REFERENCES

Braun M et al. (2016), Oncoimmunol 5(7): e1188245. doi: 10.1080/2162402X.2016.1188245
Burbach B J et al. 2007, Immunol Rev. 218: 65-81. doi: 10.1111/j.1600-065X.2007.00527.x
Cesarz Z and Tamama K, (2016), Stem cell Int. 2016:9176357. doi: 10.1155/2016/9176357
Dustin M L and Springer T A (1989), Nature 341(6243):619-24. doi: 10.1038/341619a0
Gattinoni L et al. (2011), Nat Med 341(6243):619-24. doi: 10.1038/341619a0.
Gattinoni L et al. (2017), Nat Med 23(1): 18-27. doi: 10.1038/nm.4241
Kalamasz D et al. (2004) J Immunother. 27(5):405-18. doi: 10.1097/00002371-200409000-00010
Kirkin AF et al. (2018) Nat Commun. 9(1):785. doi: 10.1038/s41467-018-03217-9
Levine B L et al. (1996) Science 272:1939-1943. doi: 10.1126/science.272.5270.1939.
Martin P J et al. (1986) J Immunol. 136:3282-3287.
Rasmussen A M et al. (2010) J Immunol Methods 355(1-2):52-60. doi: 10.1016/j.jim.2010.02.004
Rosenblatt J et al. 2010 J Immunother. 33(2):155-66. doi: 10.1097/CJI.0b013e3181bed253
Rudnicka W et al. (1992) Clin Exp Immunol. 87(1):46-52. doi: 10.1111/j.1365-2249.1992.tb06411.x
Thompson J A et al. (2003) Clin Cancer Res 9(10 Pt 1):3562-70
Van Kooyk Y et al. (1989) Nature 342(6251):811-3. doi: 10.1038/342811a0
Weinert B T. et al. (2009), Cancer Immun. 9: 9.
Zheng Q et al. (2019) Cancer Sci 111(2):383-394. doi: 10.1111/cas.14276

1. A method for preparation of a composition comprising activated human CD8+ and natural killer (NK) lymphocytes, comprising
1) isolating a sample of blood cells from a subject, wherein the sample is enriched for lymphocytes;
2) culturing a fraction of the sample in the presence of at least one agent capable of activating T lymphocytes via binding to CD3 and/or CD28 thereby stimulating proliferation of CD4⁺ lymphocytes and increasing the CD4⁺/CD8⁺ ratio compared to the lymphocytes obtained from step 1;
3) contacting the proliferating T lymphocytes with an agent that induces expression of cancer/testis antigens followed by a period of culture that results in said expression of cancer/testis antigens;
4) separating the cancer/testis antigen expressing T lymphocytes from the agent capable of activating T lymphocytes followed by mixing the cancer/testis antigen expressing lymphocytes obtained from step 3 with a second fraction of the sample from step 1; and
5) subsequently culturing the lymphocyte mixture from step 4 to stimulate proliferation of CD8⁺ and NK lymphocytes
wherein said agent comprises antibodies, antibody fragments, antibody analogues, aptamers, molecular imprinted polymers, or soluble receptors, which bind CD3 and/or bind CD28.

2. The method according to claim 1, wherein step 2 has a duration between 2 and 5 days, preferably about 3 days.

3. The method according to claim 1, wherein said second fraction of the sample is kept frozen between step 1 and until mixing in step 4.

4. The method according to claim 1, wherein the agent that induces expression of cancer/testis antigens is a DNA de-methylating agent or a histone acetylating agent.

5. The method according to claim 4, wherein the DNA de-methylating agent is selected from 5-aza-2′-deoxycytidine (5 Aza-CdR), 5-azacytidine, 5-fluoro-2′-deoxycytidine, guadecitabine, and zebularine, and wherein the histone acetylating agent is Trichostatin A or a depsipeptide.

6. The method according to claim 1, wherein the agent that induces expression of cancer/testis antigens is 5-Aza-CdR.

7. The method according to claim 1, wherein IL-2 or another agent, which stimulates proliferation of lymphocytes, is added during the course of culture of lymphocytes.

8. The method according to claim 1, wherein step 5 comprises addition of an agent capable of activating T lymphocytes via binding to CD3 and/or CD28, preferably the same type of agent as used in step 2.

9. The method according to claim 8, wherein the agent capable of activating T lymphocytes via binding to CD3 and/or CD28 is added 3-7 days after initiation of step 5, preferably after about 5 days.

10. The method according to claim 1, which is followed by isolation/recovery of the activated CD8⁺ and NK lymphocytes.

11. The method according to claim 1, wherein the agent capable of activating T lymphocytes via binding to CD3 and/or CD28 is selected from 1) an agent, comprising antibodies, antibody fragments or antibody analogues which bind CD3; 2) an agent comprising antibodies, antibody fragments or antibody analogues which bind CD28, and 3) an agent comprising antibodies, antibody fragments or antibody analogues, which bind CD3 and comprising anitbodies, antibody fragments, or antibody analogues which bind CD28.

12-13. (canceled)

14. The method according to claim 13, wherein the antibodies, antibody fragments or antibody analogues are linked to a solid or semi-solid phase, or to a polymer.

15. The method according to claim 14, wherein the solid or semi-solid phase is constituted by separable beads.

16. A method for preparation of a composition comprising activated human CD8+ and natural killer (NK) lymphocytes, comprising
a) isolating a sample of blood cells from a subject, wherein the sample is enriched for lymphocytes;
b) culturing a fraction of the sample under conditions that stimulate proliferation of CD4⁺ lymphocytes and increase the CD4⁺/CD8⁺ ratio compared to the lymphocytes obtained from step a;
c) contacting the proliferating T lymphocytes with an agent that induces expression of cancer/testis antigens followed by a period of culture that results in said expression of cancer/testis antigens;
d) separating the cancer/testis antigen expressing T lymphocytes from the agent capable of activating T lymphocytes followed by mixing the cancer/testis antigen expressing lymphocytes with a second fraction of the sample from step a; and
e) subsequently culturing the lymphocyte mixture from step 4 to stimulate proliferation of CD8⁺ and NK lymphocytes,
wherein step e) comprises addition of an agent capable of activating T lymphocytes via binding to CD3 and/or CD28, wherein said agent comprises antibodies, antibody fragments, antibody analogues, aptamers, molecular imprinted polymers, or soluble receptors, which bind CD3 and/or bind CD28.

17. The method according to claim 16, wherein the agent capable of activating T lymphocytes via binding to CD3 and/or CD28 is added 3-7 days after initiation of step 5, preferably after about 5 days.

18. The method according to claim 16, wherein the agent capable of activating T lymphocytes via binding to CD3 and/or CD28 is selected from the group consisting of 1) an agent comprising antibodies, antibody fragments or antibody analogues which bind CD28, and 3) an agent comprising antibodies, antibody fragments or antibody analogues, which bind CD3 and comprising antibodies, antibody fragments, or antibody analogues which bind CD28.

19-20. (canceled)

21. The method according to claim 16, wherein the antibodies, antibody fragments or antibody analogues are linked to a solid or semi-solid phase, or to a polymer.

22. The method according to claim 21, wherein the solid or semi-solid phase is constituted by separable beads.

23. The method according to claim 16, which is followed by isolation/recovery of the activated CD8⁺ and NK lymphocytes.

24. The method according to claim 16, wherein the conditions in step b entail co-culture with mature dendritic cells prepared from the sample in step a.

25. The method according to claim 16, wherein step c, d or e comprises addition of mature dendritic cells prepared from the sample in step a.

26. A method for treatment of cancer in a patient, comprising
1) isolating a sample of blood cells from the patient, wherein the sample is enriched for lymphocytes;
2) culturing a fraction of the sample in the presence of at least one agent capable of activating T lymphocytes via binding to CD3 and/or CD28 thereby stimulating proliferation of CD4⁺ lymphocytes and increasing the CD4⁺/CD8⁺ ratio compared to the lymphocytes obtained from step 1;
3) contacting the proliferating T lymphocytes with an agent that induces expression of cancer/testis antigens followed by a period of culture that results in said expression of cancer/testis antigens;
4) separating the cancer/testis antigen expressing T lymphocytes from the agent capable of activating T lymphocytes followed by mixing the cancer/testis antigen expressing lymphocytes obtained from step 3 with a second fraction of the sample from step 1; and
5) subsequently culturing the lymphocyte mixture from step 4 to stimulate proliferation of CD8⁺ and NK lymphocytes,
or
a) isolating a sample of blood cells from a subject, wherein the sample is enriched for lymphocytes;
b) culturing a fraction of the sample under conditions that stimulate proliferation of CD4⁺ lymphocytes and increase the CD4⁺/CD8⁺ ratio compared to the lymphocytes obtained from step a;
c) contacting the proliferating T lymphocytes with an agent that induces expression of cancer/testis antigens followed by a period of culture that results in said expression of cancer/testis antigens;
d) separating the cancer/testis antigen expressing T lymphocytes from the agent capable of activating T lymphocytes followed by mixing the cancer/testis antigen expressing lymphocytes with a second fraction of the sample from step a; and
e) subsequently culturing the lymphocyte mixture from step 4 to stimulate proliferation of CD8⁺ and NK lymphocytes, wherein step e) comprises addition of an agent capable of activating T lymphocytes via binding to CD3 and/or CD28,
and subsequently administering the cells obtained from step 4 or step e) to the patient wherein said agent comprises antibodies, antibody fragments, antibody analogues, aptamers, molecular imprinted polymers, or soluble receptors, which bind CD3 and/or bind CD28.

27. (canceled)

28. The method according to claim 26, wherein the patient receives at least or exactly 2, at least or exactly 3, or at least of exactly 4 administrations.

29. The method according to claim 26, wherein the administration is via the parenteral route.

30. The method according to claim 26, wherein the cancer is selected from the group consisting of carcinoma, adenocarcinoma, sarcoma (including liposarcoma, fibrosarcoma, chondrosarcoma, osteosarcoma, leiomyosarcoma, rhabdomyosarcoma), glioma (in particular glioblastoma), neuroblastoma, medullablastoma, malignant melanoma, neurofibrosarcoma, choriocarcinoma, myeloma, and leukemia.

31. The method according to claim 26, wherein the patient is also subjected to a co-treatment with an anticancer drug, in particular with a checkpoint inhibitor drug.

32. The method according to claim 31, wherein the co-treatment is with a PD-1 or PD-L1 inhibitor.

33. The method according to claim 31, wherein the co-treatment is prior to and/or concurrent with and/or or subsequent to the treatment defined in any one of claims 26.

34-35. (canceled)

36. The method according to claim 1, wherein the antibodies are bispecific antibodies.

37. The method according to claim 16, wherein the antibodies are bispecific antibodies.

38. The method according to claim 26, wherein the antibodies are bispecific antibodies.

39. The method according to 15, wherein the separable beads are paramagnetic or superparamagnetic beads.

40. The method according to 22, wherein the separable beads are paramagnetic or superparamagnetic beads.

41. The method according to claim 7, wherein said another agent, which stimulates proliferation of lymphocytes is selected from the group consisting of IL-15, IL-7, and IL-21 or combinations thereof.

42. The method according to claim 14, wherein the polymer is dextran.

43. The method according to claim 21, wherein the polymer is dextran.

44. The method according to claim 29, wherein the parenteral route is selected from the intraveneous route, the intraarterial route, the intratumoral route, and the intralymphatic route.
